(19) United States
(12) Patent Application Publication
(10) Pub. No.: US 2010/0322895 A1
(43) Pub. Date: Dec. 23, 2010

(54) T CELL DEPLETING COMPOSITIONS USEFUL FOR TREATING CANCER

(75) Inventor: Jason Chesney, Louisville, KY (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 12/665,795

(22) PCT Filed: Jun. 20, 2008
(86) PCT No.: PCT/US08/67730

§ 371 Date: Feb. 17, 2010

Related U.S. Application Data
(60) Provisional application No. 60/945,154, filed on Jun. 20, 2007.

(51) Int. Cl. A61K 38/20; A61K 39/395; A61K 31/519; A61K 31/675; A61K 31/137; A61K 31/255; A61K 31/7076; A61K 39/00; A61P 35/00; A61P 37/04

(52) U.S. Cl. 424/85.2; 424/173.1; 514/262.1; 514/90; 514/646; 514/517; 514/47; 424/184.1

(57) ABSTRACT

The presently-disclosed subject matter provides methods and compositions for treating or reducing the risk of recurrence of a cancer in a subject. The methods comprise administering an effective amount of a T cell depleting composition to the subject to thereby treat the cancer.

### CROSS REFERENCES TO RELATED APPLICATIONS

This application claims priority from U.S. Provisional Application Ser. No. 60/945,154 filed Jun. 20, 2007, the entire disclosure of which is incorporated herein by this reference.

### TECHNICAL FIELD

The presently disclosed subject matter relates to therapeutic methods for treating cancer in a subject. In particular, the presently disclosed subject matter relates to therapeutic methods using T cell depleting compositions for treating cancer in a subject.

### BACKGROUND

Malignant tumors, or cancers, grow in an uncontrolled manner, invade normal tissues, and often metastasize and grow at sites distant from the tissue of origin. In general, cancers are derived from one or only a few normal cells that have undergone a poorly understood process called malignant transformation. Cancers can arise from almost any tissue in the body. Those derived from epithelial cells, called carcinomas, are the most common kinds of cancers. Sarcomas are malignant tumors of mesenchymal tissues, arising from cells such as fibroblasts, muscle cells, and fat cells. Solid malignant tumors of lymphoid tissues are called lymphomas, and marrow and blood-borne malignant tumors of lymphocytes and other hematopoietic cells are called leukemias.

Cancer is one of the three leading causes of death in industrialized nations. As treatments for infectious diseases and the prevention of cardiovascular disease continues to improve, cancer is likely to become the most common fatal disease in these countries. Melanoma is one exemplary cancer exhibiting increased incidence and mortality in recent years. Melanoma incidence has risen by 25-31% over the last decade and is now the 5^{th} most common cancer in men and the 6^{th} most common cancer in women [Jemal et al. (2005)]. Further, melanoma causes a disproportionate mortality in young and middle-aged individuals and as such displays one of the highest “loss of potential life” rates among the adult-onset cancers (18.6 years per melanoma-related death) [Jemal et al. (2005)]. In the United States, over 8000 adults are expected to die of melanoma in 2007 alone, and 84% of melanoma patients with distant metastases will have succumbed to their disease 5 years from diagnosis [Jemal et al. (2005)].

Successfully treating cancer requires that all the malignant cells be removed or destroyed without killing the patient. Current methods of treating cancer continue to follow the long used protocol of surgical excision (if possible) followed by radiotherapy and/or chemotherapy, if necessary. The success rate of this rather crude form of treatment is extremely variable but generally decreases significantly as the tumor becomes more advanced and metastasizes. Further, these treatments can be associated with severe side effects including disfigurement and scarring from surgery (e.g. mastectomy or limb amputation), severe nausea and vomiting, chemotherapy, and most significantly, the damage to normal tissues such as the hair follicles, gut and bone marrow which is induced as a result of the relatively non-specific targeting mechanism of the toxic drugs which form part of most cancer treatments.

An ideal way to achieve successful cancer treatment would be to induce an immune response against the tumor that would discriminate between the cells of the tumor and their normal cellular counterparts. However, immunological approaches to the treatment of cancer have been attempted for over a century with unsustainable results. Accordingly, there is an urgent and ongoing need to develop new methods of treating cancers in a targeted manner. This notion of effective targeted killing of malignant cells has been, to date, unattainable.

### BRIEF DESCRIPTION OF THE DRAWINGS

FIGS. 1A-1C show DABIL2 (ONTAK) transiently depletes T cells in stage IV melanoma patients. Stage IV melanoma patients were administered DABIL2 (ONTAK; 12 mcg/kg daily for four days) and peripheral blood CD4+ (A), CD8+ (B) and CD4+CD8+ (C) T cells were quantified by flow cytometry (on the days indicated. 21 days after the first dose of DABIL2, the T cells had rebounded to within normal limits (see day 21). The black line is the average +/−standard deviation of all cell counts.

FIG. 2 shows that depletion of T cells induces regression of axillary lymph nodes. Stage IV melanoma patient was administered a total of 4 cycles of DABIL2 (ONTAK; cycle=12 mcg/kg daily for four days) every three weeks and bilateral axillary lymph nodes containing metastatic melanoma were measured using CT imaging.

FIG. 3 shows depletion of T cells induces regression of hepatic metastases of melanoma. Stage IV melanoma patient was administered 4 cycles of DABIL2 (ONTAK; cycle=12 mcg/kg daily for four days) every three weeks and extent of disease was quantified by PET imaging Five hepatic metastases were found to completely regress.

FIG. 4 shows depletion of T cells induces regression of pulmonary, hepatic and subcutaneous metastases of melanoma. Stage IV melanoma patient was administered 4 cycles of DABIL2 (ONTAK; 12 mcg/kg daily for four days) every three weeks and extent of disease was quantified by PET/CT imaging. Massive regression of melanoma lesions in the lungs, liver and subcutaneous space were observed.

FIG. 5 shows depletion of T cells induces infiltration of CD3+ T lymphocytes into melanoma lesions. A stage IV melanoma patient was administered 3 cycles of DABIL2 (ONTAK; cycle=12 mcg/kg daily for four days) every three weeks. After 3 cycles, a subcutaneous lesion was resected due to the possibility of severe cellulites and necrosis. Photographs (A, B), isotype control staining (C), S100 immunostaining for melanoma (E), hematoxylin/eosin staining (D) and CD3 immunostaining (F) confirmed the association between the melanoma cells and CD3+ T cells.

FIG. 6 shows depletion of T cells induces de novo induction of melanoma-specific CD8+ T cells. A stage IV melanoma patient was administered 4 cycles of DABIL2 (ONTAK; cycle=12 mcg/kg daily for four days) every three weeks. Throughout the 4 cycles, whole blood was collected and analyzed for MART1-tetramer+ CD8+ T cells using flow cytometry. Each cycle of DABIL2 is annotated by an arrow. A. Flow cytometry scatter plot revealing the de novo appearance of MART1-tetramer/CD8+ T cells after 2 cycles of DABIL2. B. Quantification of MART1+/CD8+ T cells during 4 cycles of DABIUL2.

FIGS. 7A and 7B are graphs showing DAB/IL2 transiently depletes CD4+ and CD8+ T cells in melanoma patients. 10 patients with stage IV metastatic melanoma were administered DAB/IL2 (intravenous; 12 μg/kg) daily×4 days (arrows indicate each administration). Whole blood was collected on the indicated days and analyzed for absolute lymphocyte (black), granulocyte (red) and monocyte (green) concentration with an automated hematology analyzer (A) and absolute CD4+ and CD8+ T cell concentration by flow cytometry (B). The peripheral blood concentrations of CD4+ and CD8+ T cells were quantified by multiplying the percentage of anti-CD4 or anti-CD8 fluorescence-positive cells within the lymphocyte forward/side scatter gate by the absolute lymphocyte concentration determined using an automated hematology analyzer. Percent control of each sample was calculated by dividing the absolute cell concentration on the indicated day of treatment with the absolute cell concentration on day 0 prior to DAB/IL2 administration (×100). Data are represented as averages±standard error of the mean (n=10 patients).

FIG. 8 is a series of graphs showing DAB/IL2 transiently depletes CD4+/CD25HI/Foxp3+ T cells. Whole blood was collected from patient P9 during cycle one of DAB/IL2 administration just prior to the first (day 0) and last dose (day 3) and then 7 and 21 days after initiation of DAB/IL2 therapy. The peripheral blood mononuclear cells were isolated from the whole blood by Ficoll gradient centrifugation and stained with fluorescent conjugates of monoclonal antibodies specific for CD4, CD25 and Foxp3. In order to quantify the percentage of CD4+/CD25HI/Foxp3+ T cells within the total lymphocyte forward/side scatter gate, the CD4+/CD25HI cells (right panels) were gated and analyzed for Foxp3 expression (left panels).

FIGS. 9A and 9B are graphs showing DAB/IL2 transiently depletes all analyzed CD4+ T cell subsets. Whole blood was collected from 10 patients throughout the first cycle of DAB/IL2 and analyzed for CD4+/CD25HI/Foxp3+ co-expression by flow cytometry as described in the FIG. 8 legend and Methods section of Examples 5-7. The absolute concentration of CD4+/CD25-(black), CD4+/CD25+ (red) and CD4+/CD25HI (green) T cells (A) and of CD4+/CD25HI/Foxp3− (black) and CD4+/CD25HI/Foxp3+ (red) T cells (B) were quantified by multiplying the percentage of anti-CD4, anti-CD25 and/or anti-Foxp3 fluorescence-positive cells within the lymphocyte forward/side scatter gate by the absolute lymphocyte concentration determined using an automated hematology analyzer. The percent control of each sample was calculated by dividing the absolute cell concentration on the indicated day of treatment with the cell concentration on day 0 prior to DAB/IL2 administration (×100). Data are represented as averages±standard error of the mean (n=10 patients).

FIGS. 10A-10H are graphs showing reduction in the T cell depleting activity of DAB/IL2 during cycles 2-4 is associated with the development of anti-DAB/IL2 IgG. Whole blood was collected on the indicated days from patients P3 (A, B), P7 (C, D), P9 (E, F) and P16 (G, H) throughout four cycles of DAB/IL2 administration (each cycle indicated by an arrow). CD4+ (black), CD8+ (red), CD4+/CD25HI/Foxp3− (green) and CD4+/CD25HI/Foxp3+ (purple) T cells and monocytes (blue) were quantified as described in FIGS. 7-9 (A, C, E, H). Percent control of each sample was calculated by dividing the absolute cell concentration on the indicated day of treatment with the cell concentration on day 0 prior to DAB/IL2 administration (×100). Plasma was isolated on the indicated days and analyzed for the presence of anti-DAB/IL2 IgG by ELISA. For the ELISA, data are presented as averages±standard deviations (n=5 per sample).

FIG. 11 is a series of flow cytometric scatter plots demonstrating the de novo appearance of MART1-, gp100- and tyrosinase-specific CD8+ T cells after one cycle of DAB/IL2. Whole blood was collected from patient P16 during cycle one of DAB/IL2 administration just prior to (day 0) and 21 days after the first dose of DAB/IL2. The peripheral blood mononuclear cells were isolated from the whole blood by Ficoll gradient centrifugation and stained with a PE-labeled anti-CD8 monoclonal antibody and the indicated APC-labeled tetrameric HLA-A2*0201/peptide conjugates.

FIGS. 12A-12D is a series of graphs showing de novo appearance of MART1-, gp100- and/or tyrosinase-specific CD8+ T cells in 4/7 HLA-A2*0201+ melanoma patients after one cycle of DAB/IL2. Whole blood was collected from patients P7 (A), P9 (B), P14 (C) and P16 (D) throughout four cycles of DAB/IL2 administration (each cycle indicated by an arrow). The peripheral blood mononuclear cells were isolated from the whole blood by Ficoll gradient centrifugation and stained with a PE-labeled anti-CD8 monoclonal antibody and APC-labeled tetrameric HLA-A2*0201/MART1 (black) or gp100 (red) or tyrosinase (green) peptide conjugates. The peripheral blood concentration of the indicated melanoma antigen-specific CD8+ T cells was quantified by multiplying the percentage of CD8+/tetramer+ cells within the lymphocyte forward/side scatter gate by the absolute lymphocyte concentration determined using an automated hematology analyzer. Data are represented as averages±standard error of the mean (n=10 patients). Patient P14 did not develop detectable tyrosinase- or gp100-specific CD8+ T cells but the green line (tyrosinase) is concealing the red line (gp100) (C).

FIGS. 13A and 13B are photographs showing Regression of hepatic, mesenteric and hilar melanoma metastases after DAB/IL2 administration. A. Patient P3 was scanned by combination PET/CT imaging 2 weeks prior to DAB/IL2 administration (pre-DAB/IL2) and after completing four 3-week cycles of DAB/IL2 (post-DAB/IL2). The brain, heart and bladder have normal accumulations of the PET tracer 18F-fluorodeoxyglucose but several areas of increased metabolism consistent with melanoma metastases resolved after DAB/IL2 administration. B. CT imaging of patient P5 revealed a large right hilar mass and a mesenteric mass that both decreased in size after DAB/IL2 administration.

FIGS. 14A and 14B are a series of photographs showing regression of subcutaneous, intramuscular and lymphatic metastases after DAB/IL2 administration. A. The right lower extremity of patient P8 was scanned by CT imaging 3 weeks prior to DAB/IL2 administration (pre-DAB/IL2) and after completing four 3-week cycles of DAB/IL2 (post-DAB/IL2). The white numbers in the lower left corner of each image indicate the distance (mm) above the superior aspect of the patella in order to provide matched images for comparison. B. CT imaging of patient P9 revealed a rapidly growing right inguinal mass that decreased in size 3 months after DAB/IL2 administration. A follow-up scan, 6 months after DAB/IL2 administration, revealed no further growth.

FIG. 15 is a series of photographs showing stabilization of two right hilar masses in a 79-year old male after DAB/IL2 administration. PET imaging of patient P12 was conducted 1 month prior and 3 months after 2 cycles of DAB/IL2. Two discrete areas of hypermetabolism in the right hilum remained stable during this three month period.

FIGS. 16A-16D are a series of photographs showing near complete response of widespread visceral melanoma metastases after 4 cycles of DAB/IL2. A. Anterior/posterior views, PET. B. Lateral views, PET. C. CT imaging, liver. D. CT imaging, lungs. Combined PET/CT imaging of patient P14 revealed rapid progression of multiple melanoma metastases in the liver, both lungs, lymph nodes and the subcutaneous compartment (compare −6 months to −1 week). After 4 cycles of DAB/IL2, the liver metastases completely resolved and the lung metastases markedly regressed (compare −1 week to +3 months). Three months after completion of DAB/IL2, the residual lung metastases had completely resolved but a single enlarged peri-aortic lymph node persisted (red arrow). The increased 18F-fluorodeoxyglucose uptake in the brain, bladder and both kidneys are due to normal metabolism and are not reflective of metastases.

FIGS. 17A-17C are a series of photographs showing CD8+ T cell infiltration of residual HLA-A, B and C negative melanoma and evidence for vitiligo after DAB/IL2 administration. The residual peri-aortic mass in patient P14 was resected, formalin fixed and embedded in paraffin. A. Hematoxylin/eosin (H&E) staining of the mass revealed a mononuclear infiltrate that was confirmed to include CD8+ T cells by double immunohistochemistry using an anti-CD8 antibody (brown) and an anti-MART1 antibody (red). The counter stain used in the immunohistochemistry was hematoxylin and the control consisted of no primary antibody. B. HLA-A, B or C expression by cells in the pancreas (top; positive control), an unrelated melanoma metastasis (middle; positive control) and the residual peri-aortic melanoma metastasis resected from patient P14 as determined using a monoclonal antibody specific for a non-polymorphic portion of these HLA molecules. C. Photographs of patient P14's hair before and after DAB/IL2 administration revealed the complete loss of pigmentation.

### DETAILED DESCRIPTION

The details of one or more embodiments of the presently disclosed subject matter are set forth in the accompanying description below. Other features, objects, and advantages of the presently disclosed subject matter will be apparent from the detailed description, figures, Appendix, and claims. All publications, patent applications, patents, and other references referenced herein are incorporated by reference in their entirety. In case of conflict, the present specification, including definitions, will control.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The presently disclosed subject matter provides in some embodiments, methods of stimulating an immune response against a cancer in a subject so as to facilitate a targeted attack of the cancer by the subject's immune system. In some embodiments, the methods comprise depleting T lymphocytes (“T cells”) in the subject and then permitting rebounding of T cells in the subject to thereby stimulate an immune response against the cancer. In some embodiments, the methods further comprise repeating the depleting and the permitting of rebounding of T cells in the subject a desired number of times. Although the amount of depletion that enables induction of tumor-specific immunity and tumor regressions has been found to be between 50-90%, T cell depletion of as little as 10% is expected to have beneficial anti-tumor effects. In some embodiments, depleting T cells in the subject can be achieved by administering an effective amount of a T cell depleting composition to the subject.

By “rebounding of T cells”, it is meant that the population of functioning T cells within the subject increases over time as compared to the depleted state. The T cell population in the subject can in some embodiments return to viable count levels equivalent to T cell counts prior to depleting the T cells. However, the term “rebounding of T cells” is further inclusive of an increase in T cell counts, but not a complete return of T cell counts to pre-depletion levels. Further, the term “rebounding of T cells” is intended to be inclusive of an increase in certain subclasses of T cells, but not necessarily all subclasses. In particular, and without wishing to be bound by any particular theory of operation, the data presented herein in the Examples and Appendix indicate that depletion of T cell populations, including total T cell populations, can facilitate improved T cell-mediated tumor rejection by reducing tumor tolerance barriers and providing for a “fresh look” at the tumor by the immune system, which can stimulate the proliferation of T cells active against cancer cells, including antitumor CD8⁺ T cells. Thus, in some embodiments, depleting T cells comprises depleting total T cell counts in the subject. Further, rebounding of T cell counts can in some embodiments include the rebounding of particular subpopulations (e.g., antitumor CD8⁺ T cells) to a greater extent than other populations.

The presently disclosed subject matter further provides in some embodiments, methods of treating or reducing the risk of recurrence of a cancer in a subject. In some embodiments, the methods comprise administering an effective amount of a T cell depleting composition to the subject.

“Treating a cancer” refers to inhibiting or preventing oncogenic activity of cancer cells. Oncogenic activity can comprise inhibiting migration, invasion, cell survival, anchorage-independent growth, angiogenesis, or combinations thereof of the cancer cells.

The terms “cancer” and “cancer cell” are used interchangeably herein and refer generally to a group of diseases characterized by uncontrolled, abnormal growth of cells (e.g., a tumor). In some forms of cancer, the cancer cells can spread locally or through the bloodstream and lymphatic system to other parts of the body (“metastatic cancer”).

As used herein, “cancer” refers to all types of cancer or neoplasm or malignant tumors found in animals, including leukemias, carcinomas and sarcomas. Examples of cancers are cancer of the brain, bladder, breast, cervix, colon, head and neck, kidney, lung, non-small cell lung, melanoma, mesothelioma, ovary, prostate, sarcoma, stomach, uterus and Medulloblastoma.

By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the present methods, the cancer treated is a melanoma. The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The presently disclosed subject matter provides for the treatment of a cancer and stimulating an immune response against a cancer by administering to the subject a T cell depleting composition. A “T cell depleting composition”, as used herein, is a composition that can reduce numbers of viable (i.e., biologically active) T cells (“T cell counts”) within a subject for at least a transient period of time. T cell depleting compositions can be cytotoxic for T cells. Exemplary T cell depleting compositions include, but are not limited to, methotrexate, busulfan, cyclophosphamide, fludarabine, FTY720, anti-CD3 antibodies such as muromonab-CD3 (e.g., ORTHOCLONE OKT® 3, Ortho Biotech Products, Bridgewater, N.J.); and hOKT3γ1(Ala-Ala)), IL-2-cell toxin fusion proteins, or combinations thereof.

Exemplary IL-2-cell toxin fusion proteins that can be utilized with the present methods as T cell depleting compositions include, but are not limited to, IL-2-cell toxin fusion proteins wherein the cell toxin is a diphtheria toxin. For example, in some embodiments of the presently disclosed subject matter, the T cell depleting composition utilized is DAB₃₈₉IL-2 (also referred to as denileukin diftitox and “ONTAK”®, Ligand Pharmaceuticals Incorporated, San Diego, Calif.). DAB₃₈₉IL-2 is a recombinant DNA-derived cytotoxic fusion protein composed of the amino acid sequences for diphtheria toxin fragments A and B (Met1-Thr387)-His followed by the sequences for interleukin-2 (IL-2; Ala1-Thr133). DAB₃₈₉IL-2 is designed to direct the cytocidal action of diphtheria toxin to cells which express the IL-2 receptor, including T cells. Ex vivo studies suggest that DAB₃₈₉IL-2 interacts with the high affinity form of IL-2 receptors on the cell surface and inhibits cellular protein synthesis, resulting in death of cells expressing the IL-2 receptors within hours.

Suitable methods for administering to a subject a therapeutic compound in accordance with the methods of the present subject matter include but are not limited to systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance compound accumulation at a target site (see, e.g., U.S. Pat. No. 6,180,082). The particular mode of administration used in accordance with the methods of the present subject matter depends on various factors, including but not limited to the compound and/or carrier employed, the severity of the condition to be treated, and mechanisms for metabolism or removal of the compound following administration.

A therapeutic T cell depleting composition as described herein can further include a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The T cell depleting compositions used in the present methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods known in the art. For example, a T cell depleting composition disclosed herein can be formulated as a pH stabilized core having an enteric or delayed release coating which protects the T cell depleting composition until it reaches the desired location in the gastrointestinal tract.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

The term “effective amount” is used herein to refer to an amount of the therapeutic T cell depleting composition sufficient to produce a measurable biological response (e.g., a reduction in T cells, as disclosed herein above). Actual dosage levels of active ingredients in a therapeutic composition of the presently disclosed subject matter can be varied so as to administer an amount of the active compound(s) that is effective to achieve the desired therapeutic response for a particular subject and/or application. The selected dosage level will depend upon a variety of factors including the activity of the therapeutic composition, formulation, route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art of medicine.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For parenteral administration, in some embodiments, the T cell depleting composition comprises DAB₃₈₉IL-2 and can be employed in an effective amount ranging from about 9 mcg/kg to about 18 mcg/kg to achieve the desired T cell depletion. In some particular embodiments, the T cell depleting composition comprises DAB₃₈₉IL-2 and is administered intravenously at a dosage of about 12 mcg/kg. Further, in some particular embodiments, the T cell depleting composition comprises DAB₃₈₉IL-2 and is administered intravenously in a cycle of once daily for four days, and wherein the cycle is repeated about every 21 days for at least three cycles. Additional cycles have been found to be beneficial in a subset of patients and therefore the period of efficicacy of the T cell depletion strategy to induce tumor-specific immunity and tumor regression can be extended throughout the lifetime of a cancer patient, if desirable.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et at, (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzunq, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

With respect to the therapeutic methods of the presently disclosed subject matter, a “subject” as the term is used herein in some embodiments refers to a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

### Examples

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

Several different types of cancer are believed to be particularly well-suited for immunological intervention therapies. For example, melanoma is generally held to be amenable to immunological intervention. This perception is based on the following: (i) several melanoma-specific antigens have been identified; (ii) melanoma antigen-specific CD4+ and CD8+ T lymphocytes are present in melanoma patients and have anti-tumor activity; (iii) immune-enhancing agents can cure mice of established melanomas; and (iv) spontaneous regressions in humans with concurrent onset of vitiligo have been reported [Jemal et al. (2005) and Thompson et al. (2005)]. Significantly, in patients with intermediate or high risk of recurrence, the immune-enhancing agent, interferon-alpha, increases survival, and high-dose IL-2, a potent stimulator of T cell proliferation, causes durable remissions in a small subset of patients with metastatic melanoma [Atkins et al. (1999), Keilholz et al. (1998) and Kirkwood et al. (1996)].

In the present Examples, the effect of an exemplary T cell depleting composition, DAB₃₈₉IL-2 on tumor volume in ten patients with Stage IV melanoma was examined. Five patients experienced significant regression of several metastatic tumors. A significant decrease in total T cell counts and a rebound increase in melanoma-specific CD8+ T cells after DAB₃₈₉IL-2 administration was also observed. One patient required resection of an inflamed subcutaneous melanoma lesion and immunohistochemical analysis revealed the presence of CD3+ T cells within a mononuclear cell infiltration of the tumor.

### Materials and Methods for Examples 1-4

Patient Enrollment

This clinical trial was approved by the University of Louisville Human Studies Committee. Only patients with distant metastases from cutaneous or mucosal melanoma or melanoma of unknown primary were eligible for inclusion. All patients fulfilled the following criteria: (i) primary tumor must have been documented by histopathologic analysis; (ii) metastatic disease must have been documented by radiologic examinations (CT scan or PET scan) with bidimensional measurements; (iii) disease recurrences occurring greater than five years after the original diagnosis must have been biopsy proven and; (iv) patients with lymph node metastases in multiple lymph node beds who were not amenable to surgical resection were included in this study—those patients with involvement of a single lymph node bed were not eligible.

DAB₃₈₉IL-2 Administration

All patients were subjected to fusion PET/CT or CT imaging within one month prior to receiving the first dose of DAB₃₈₉IL-2 and within one month after receiving the last dose of ONTAK. DAB₃₈₉IL-2 was administer as follows: one cycle=12 mcg/kg, IV over 30 minutes daily for 4 days, 3-4 cycles every 21 days.

All patients had renal function tests, blood counts, and a thorough physical examination, including neurological examination, prior to each cycle of DAB₃₈₉IL-2. The endpoint definitions were as follows:

Clinical Complete Response (CR)

Disappearance of all evidence of tumor. The patient must be free of all symptoms of cancer.

Partial Response (PR)

30% or greater decrease in the sum of the longest diameter of target lesions, taking as reference the baseline sum longest diameter.

Progressive Disease (PD)

At least 20% increase in the sum of the longest diameter of target lesions, taking as reference the baseline sum longest diameter, or the appearance of new lesions and/or unequivocal progression of existing non-target lesion

Stable Disease (SD)

Neither sufficient shrinkage to qualify for partial response nor sufficient increase to qualify for progressive disease, taking as reference the smallest sum longest diameter since the treatment started.

Peripheral Blood MART-1, gp100 and Tyrosinase-Specific CD8+ T Cell Enumeration

PBMCs were isolated from the collected whole blood by centrifugation through Accuspin System Histopaque 1077 and then washed twice with PBS. For staining, 10⁶ PBMCs were incubated at 37° C. for 30 minutes in the dark with 0.5 to 1.0 μg of APC-labeled tetramer (MART-1, gp100 or tyrosinase, Immunomics by Beckman Coulter), then with a CD8-PE monoclonal antibody (R&D Systems) for 15 minutes at 4° C. Small lymphocytes were gated according to forward/side-scatter profiles, then CD8^{high} cells were selected, and staining with 7AAD (BD PharMingen) was used to exclude dead cells. Data was collected on a FACSCalibur flow cytometer within 1 hour after staining, and then analyzed with Cell Quest software (Becton Dickinson).

Histology

Five-micrometer sections of formalin-fixed and paraffin-embedded tumor tissues were treated with xylene to remove paraffin and then rehydrated. Hematoxylin-eosin staining for collagen were performed using standard procedures. For immunohistochemical staining, deparaffinized and rehydrated sections were blocked by incubation with serum blocking buffer for 30 minutes at room temperature. Tissue sections were incubated for 1 hour with rabbit anti-S100 antibody or mouse anti-CD3 for the detection of melanoma cells and T cells, respectively. The sections were then incubated with biotinylated goat anti-rabbit or rabbit anti-mouse IgG for 30 minutes and developed with an avidin-biotin peroxidase reaction using 3,3′-diaminobenzidine tetrahydrochloride as chromogen. After counterstaining with Mayer's hematoxylin, the sections were dehydrated and coverslips were attached with Permount. Appropriate negative controls (by omission of the primary antibody) were used.

### Example 1

### DAB₃₈₉IL-2 Decreases CD4+ and CD8+ T Cells

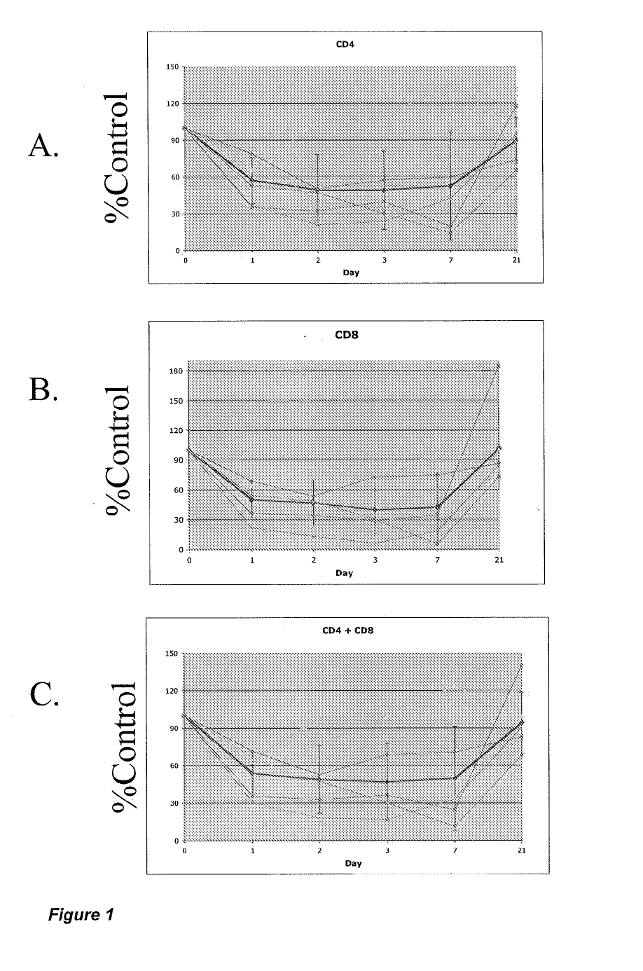

We administered DAB₃₈₉IL-2 (12 mcg/kg daily×four days) to several stage IV melanoma patients and measured the peripheral blood concentration of CD4+ and CD8+ T cells just prior to and 3 days after the four doses of DAB₃₈₉IL-2. After four doses of DAB₃₈₉IL-2, we observed a large decrease in peripheral blood CD4+ (FIG. 1A), CD8+ (FIG. 1B) and total T cells (FIG. 1C) in each patient examined. These data indicate that DAB₃₈₉IL-2 can deplete peripheral blood CD4+ and CD8+ T cells in humans. We also found that the peripheral blood T cell concentration had rebounded to within normal limits 21 days after the first dose of DAB389IL-2.

### Example 2

### DAB₃₈₉IL-2 Decreases Tumor Burden in Stage IV Melanoma Patients

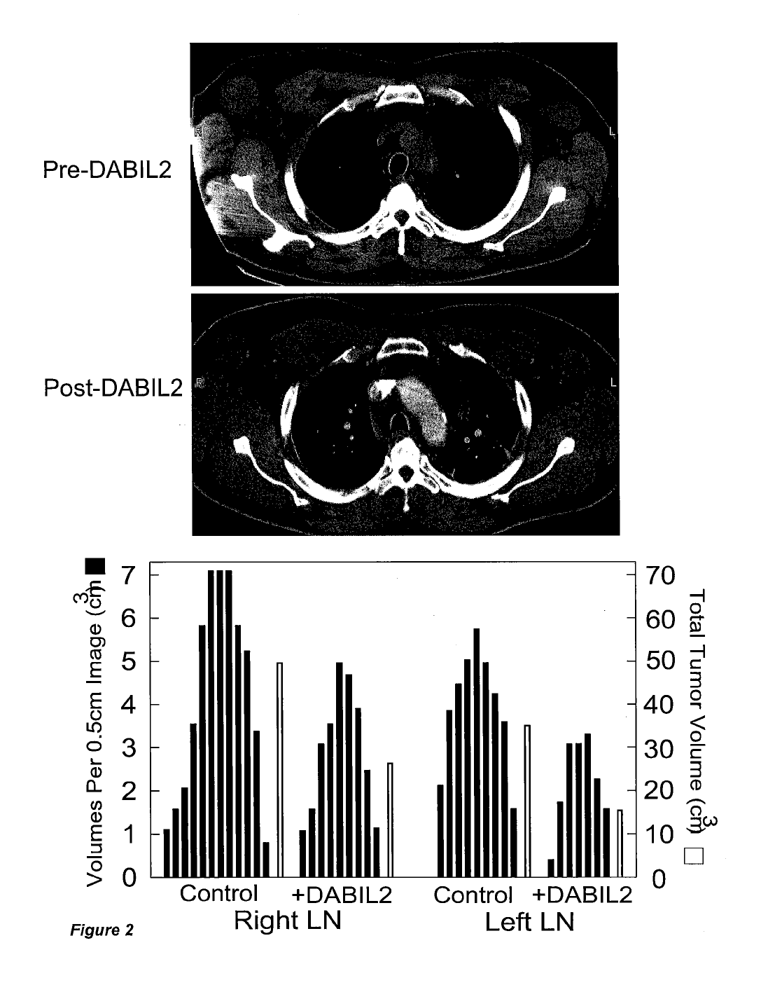

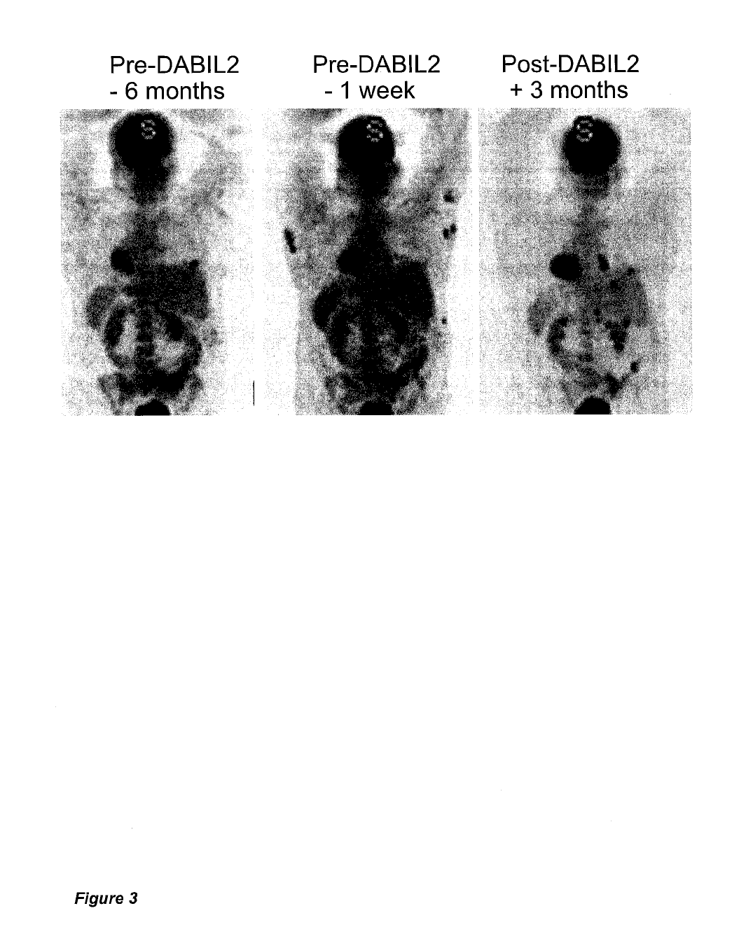

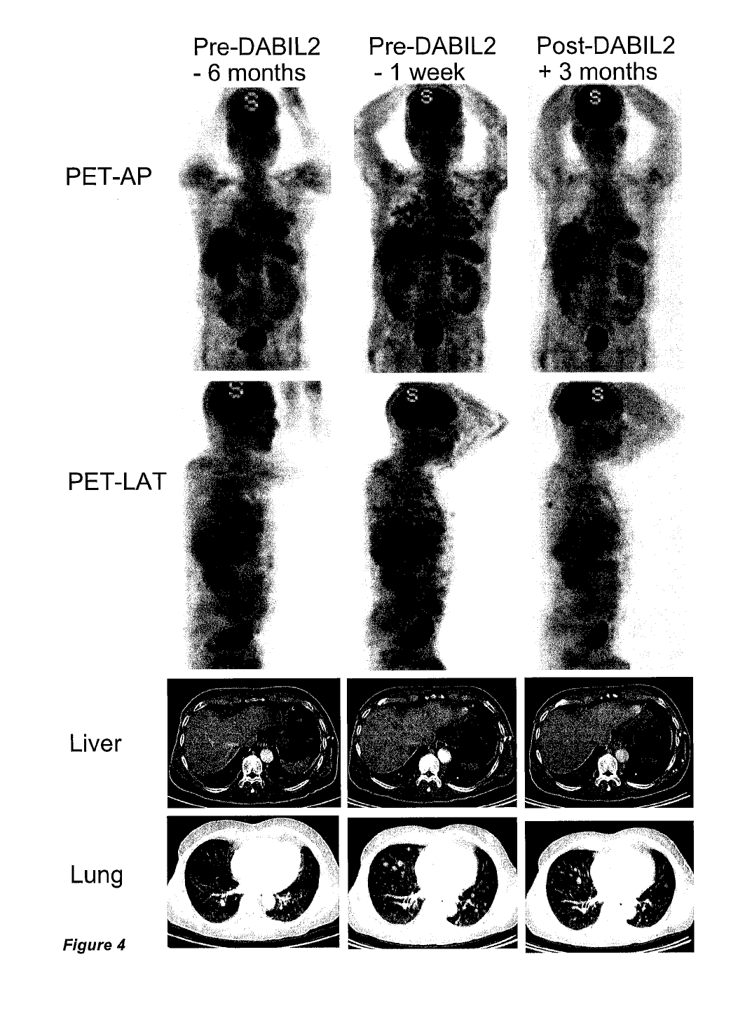

Ten heavily pre-treated stage IV melanoma patients were administered 3-4 cycles of DAB₃₈₉IL-2 (12 mcg/kg daily×four days every 3 weeks). Positron emission tomography or computed tomography were used to evaluate the patients' baseline tumor burden and potential responses three months from initiation of therapy. Table I details the characteristics of the patients and the observed responses. We observed marked reductions in visceral tumor burden in three patients, subcutaneous melanoma regressions in two patients and stabilization of tumor burden in one patient. Patient DI-1 developed rapidly progressing axillary lymph nodes and was treated with 4 cycles of DAB₃₈₉IL-2. After 3 months, a repeat chest CT revealed a large decrease in tumor volume (FIGS. 2A and 2B). Patient DI-2 also had rapidly progressed over 3 months with new and enlarging hepatic melanoma metastases (compare PET scans, FIG. 3). After 4 cycles of DAB₃₈₉IL-2, 5 distinct hepatic metastases were undetectable by PET imaging (FIG. 3). Patient DI-9 presented to our institution with widespread disease involving the lungs, liver, subcutaneous compartment and adrenal glands (FIG. 4). Clinically, he was suffering from appetite and weight loss, fatigue, nausea and shortness of breath. After four cycles of DAB₃₈₉IL-2, PET/CT imaging revealed the complete regression of all hepatic metastases and the majority of pulmonary nodules (FIG. 4). His symptoms completely resolved. Taken together, these three responses suggest that T cell depletion may have clinical utility in the treatment of melanoma.

TABLE I
Summary of Responses To DAB₃₈₉IL-2

| Patient | Age/Sex | Disease Sites | Prior Rx | Cycles | Response |
|---|---|---|---|---|---|
| 1 | 54/M | LN, SC | BC, T | 4 | PR (LN) |
| 2 | 46/M | Liver | IL2, BC 4 | | PR (LIVER) |
| 3 | 76/M | SC, Lung | T | 3 | PD |
| 4 | 77/M | Lung | T | 4 | SD |
| 5 | 37/M | SC, LN | IL2, BC, T | 4 | PD |
| 6 | 73/M | SC | T | 4 | PD |
| 7 | 69/M | Lung, Liver | T | 4 | PD |
| 8 | 49/F | SC, LN | T, IFN | 3 | PR (SC) |
| 9 | 54/M | Lung, Liver, | | 4 | PR (Lung, |
| | | SC BC | | | Liver, SC) |
| 10 | 52/M | LN | IL2, BC | 4 | PR |

BC Biochemotherapy
IL2 High Dose IL2
T Temodar
LN Lymph Node
SC Subcutaneous
PD Progressive Disease
PR Partial Response
SD Stable Disease
IFN Interferon

### Example 3

### DAB₃₈₉IL-2 Caused Inflammation and Mononuclear Cell Infiltration of a Subcutaneous Melanoma Lesion

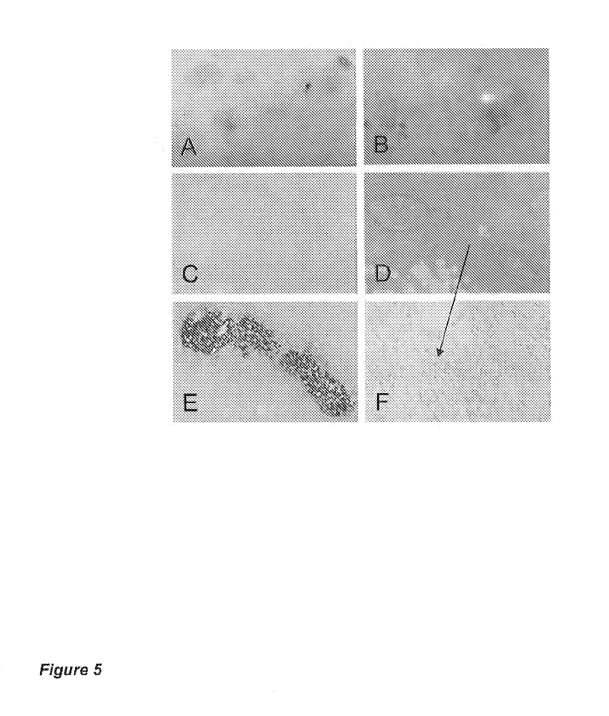

In the first stage IV melanoma patient treated with DAB₃₈₉IL-2 at our institution, we observed overt signs of inflammation associated with subcutaneous melanoma lesions after two cycles of DAB₃₈₉IL-2 (FIGS. 5A and 5B). One of these tumors appeared necrotic with an associated abscess and required resection of the tumor (FIG. 5B). Immunohistochemical analysis for the melanoma-specific protein S100 highlighted the location of this tumor and adjacent sections were stained with hematoxylin and eosin. We found that the neoplastic cells of the inflamed melanoma lesion were pyknotic and surrounding by a mononuclear cells (FIGS. 5D and 5E). An adjacent section was stained for the T lymphocyte specific protein, CD3, and T lymphocytes were identified within this mononuclear infiltrate (FIG. 5F). The clinical requirement for resection was fortuitous in that these data support the role of the cognate immune system in the efficacy of DAB₃₈₉IL-2 in stage IV melanoma.

### Example 4

### DAB₃₈₉IL-2 Increased MART1-Specific CD8+ T Lymphocytes in a Stage IV Melanoma Patient

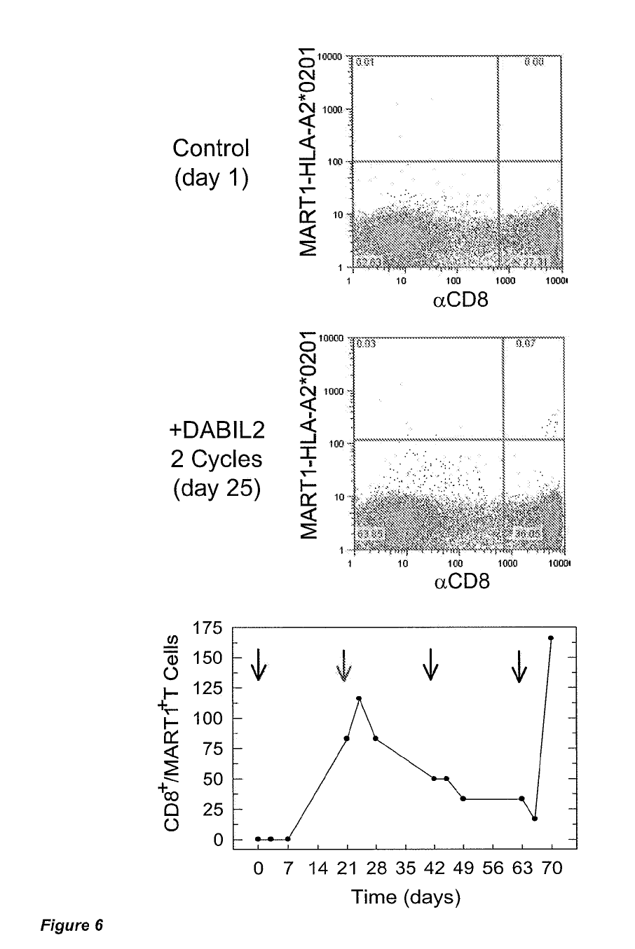

In two patients positive for the class I MHC encoded by HLA-A-0201+, we examined the peripheral blood for CD8+ T lymphocytes specific for the melanocyte differentiation antigens, MART1, tyrosinase and gp100 using tetramers. Although, we did not observe any increase in tyrosinase or gp100 specific CD8+ T cells in either patient, we did observe a marked increase in the peripheral blood concentration of MART1-specific CD8+ T cells in patient DI9 and DI-10 after only 3 DAB₃₈₉IL-2 doses (FIG. 6 for patient DI-9).

In conclusion, we have demonstrated that selective T cell depletion in melanoma patients using T cell depleting compositions allows the induction of melanoma-specific immunity and the regression of melanoma metastases. A rebound proliferation occurs after the T cell depletion within 2-3 weeks, which, without wishing to be bound by theory, can allow for the preferential expansion of T cells which are specific against melanoma cells. T cells regulate and effect immunity against neoplastic cells and have the potential to cure patients suffering from cancer. We have found that depletion of T cells caused a CD3+ T cell infiltrate in one patient and de novo induction of melanoma-specific CD8+ T cells in two other patients. We have demonstrated that specific T cell depletion is an effective approach to causing tumor regression in subjects. Several agents are available for clinical use that have previously been found to deplete T cells but none have been examined in this context. Importantly, the approach of T cell depletion is not melanoma-specific as the immune system has the potential to become activated against all types of cancer.

### Materials and Methods for Examples 5-7

Patient Enrollment

This clinical trial was approved by the University of Louisville Human Studies Committee. Only patients with distant metastases from cutaneous or mucosal melanoma or melanoma of unknown primary were eligible for inclusion. All patients fulfilled the following criteria: (i) primary tumor must have been documented by histopathologic analysis; (ii) metastatic disease must have been documented by radiologic examinations (CT scan or PET scan) with bidimensional measurements; and (iii) disease recurrences occurring greater than five years after the original diagnosis must have been biopsy proven.

DAB/IL2 (ONTAK) Administration

All patients were subjected to fusion PET/CT or CT imaging within one month prior to receiving the first dose of DAB/IL2 and within one month after receiving the last dose of DAB/IL2. DAB/IL2 was administered as follows: 12 μg/kg, IV over 30 minutes every 24 hours for 4 doses (cycles repeated every 21 days). All patients had renal function tests, blood counts, and a thorough physical examination, including neurological examination, prior to each cycle of DAB/IL2. The endpoint definitions were as follows:

Clinical Complete Response (CR)

Disappearance of all evidence of tumor. The patient must be free of all symptoms of cancer.

Partial Response (PR)

30% or greater decrease in the sum of the longest diameter of target lesions, taking as reference the baseline sum longest diameter.

Progressive Disease (PD)

At least 20% increase in the sum of the longest diameter of target lesions, taking as reference the baseline sum longest diameter, or the appearance of new lesions and/or unequivocal progression of existing non-target lesion.

Stable Disease (SD)

Neither sufficient shrinkage to qualify for partial response nor sufficient increase to qualify for progressive disease, taking as reference the smallest sum longest diameter since the treatment started.

Monocyte, Granulocyte, Lymphocyte and T Cell Subset Quantification

Whole blood (50 ml) was collected in heparinized tubes and the absolute lymphocyte, granulocyte and monocyte peripheral blood concentrations were determined with a Sysmex XE-2100 Automated Hematology Analyzer. PBMCs were then isolated by centrifugation through Accuspin System Histopaque 1077 and washed twice with PBS.

In order to determine the percentage of CD4⁺, CD4⁺/CD25⁻, CD4⁺/CD25⁺, CD4⁺/CD25″¹, CD4⁺/CD25^{HI}/Foxp3⁻, and CD4⁺/CD25^{HI}/Foxp3⁺T cells within the lymphocyte gate (based on forward/side scatter profile), we incubated the total PBMCs with PE-anti-Foxp3, FITC-anti-CD4, and APC-anti-CD25 (eBioscience). 100 μl of PBMCs (1×10⁶) were added to 20 μl of an anti-CD4/and-CD25 cocktail (1 μg anti-CD4 and 0.125 μg anti-CD25; eBioscience) and incubated for 30 minutes in the dark at 4° C. and then washed in cold PBS. After decanting, the cell pellet was resuspended in residual buffer and 1 ml of freshly prepared eBioscience Fixation/Permeabilization Buffer was added to each sample and incubated at 4° C. for 60 minutes in the dark. 2 ml of Permeabilization Buffer was used for washing followed by centrifugation and decanting of supernatant. 20 μl anti-human Foxp3 (PCH101) antibody or 20 μl rat IgG2b isotype control was added to resuspended cells and incubated at 4° C. for 30 minutes in the dark. Cells were washed twice in 2 ml Permeabilization Buffer. Small lymphocytes were gated according to forward/side-scatter profiles and data was collected on a FACSCalibur flow cytometer within 1 hour after staining, and then analyzed with Cell Quest software (Becton Dickinson).

In order to detect the percentage of total CD8⁺ cells, and MART1-, gp100- and tyrosinase-specific CD8+ T cells within the lymphocyte gate (based on forward/side scatter profile), 10⁶ PBMCs in 200 μl of flow cytometry staining buffer were incubated at 25° C. for 30 minutes in the dark with 1.0 μg of APC-labeled tetramer (MART-1, gp100 or tyrosinase; Immunomics, Beckman Coulter) and 0.25 μg CD8-PE monoclonal antibody (R&D Systems). Small lymphocytes were gated according to forward/side-scatter profiles and then the percentage of tetramer⁺CD8⁺ cells was determined. Data was collected on a FACSCalibur flow cytometer within 1 hour after staining, and analyzed with Cell Quest software (Becton Dickinson).

The absolute concentrations of CD4⁺, CD4⁺/CD25⁻, CD4⁺/CD25⁺, CD4⁺/CD25^{HI}, CD4⁺/CD25^{HI}/Foxp3⁻, CD4⁺/CD25^{HI}/Foxp3⁺, CD8⁺, CD8⁺/HLA-A2*0201-MART1-binding, CD8⁺/HLA-A2*0201-gp100-binding and CD8⁺/HLA-A2*0201-tyrosinase-binding cells were quantified by determining the percentage of fluorescence-positive cells within the forward/side scatter lymphocyte gate (as detailed above), and then multiplying this percentage by the absolute lymphocyte concentration determined using the Sysmex XE-2100 Automated Hematology Analyzer. The percent control of each sample was calculated by dividing the T cell subset absolute cell concentration on the indicated day of treatment with the cell concentration on day 0 prior to DAB/IL2 administration (×100).

DAB/IL2 Enzyme Linked Immunosorbent Assay

Human plasma samples were tested for the presence of IgG specific for DAB/IL2 by enzyme linked immunosorbent assay (ELISA). The assay was carried out as follows: 96-well microtest polystyrene assay plates (BD) were coated (100 μL/well) with either Tris-NaCl pH 8.5 solution (30 mL 5 M NaCl+50 mL 1 M Tris+920 mL water) or DAB/IL2 (Ligand) diluted to 2 μg/mL in Tris-NaCl solution. After incubating overnight at 37° C., the plates were washed two times with Tris-NaCl solution. 300 μl PBS/BSA (30 mL PBS+300 mg BSA; Sigma) was then added to each well and the plates were incubated for one hour at 37° C., followed by three washes with Tris-NaCl solution. 100 μl of test sera, diluted 1:500 in PBS/BSA solution, was then added to each well. After incubating at 37° C. for two hours, the plates were washed three times with Tris-NaCl+0.05% Tween (300 mL Tris-NaCl+150 μl Tween). 100 μl of rabbit anti-human IgG HRP-conjugated antibody (Pierce), diluted 1:50,000 in PBS/BSA, was then added to each well and the plates incubated at 37° C. for one hour, followed by three washes with Tris-NaCl+0.05% Tween and two washes with DH₂O. 100 μl of TMB substrate (Pierce) was added to each well. After five minutes, the reaction was stopped with 1N HCL (100 μl/well) and the plates were read at 450 nm.

Immunohistochemistry

Five μm sections of formalin-fixed and paraffin-embedded tumor tissue were mounted on charged glass slides and dried at 58° C. for 60 minutes. Slides were first deparaffinized with xylene then incubated with a high temperature epitope retrieval solution (20 min) and hydrogen peroxide (H₂O₂) (for 10 min) to block endogenous peroxidases. The sections were incubated with primary antibody (anti-CD8, 1:50, Dako; anti-CD4, 1:50, Novocastra; anti-HLA Class I [HLA-A, B, C], 1:500, clone EMR8-5, MBL International) for 15 min, followed by a post-primary antibody and a polymer horse-radish-peroxidase linked detection system (each for 8 min, Define, Leica Microsystems). The sections were developed with 3,3′-diaminobenzidine tetrahydrochloride (DAB) solution (Invitrogen) for 10 min and nuclei counterstained with hematoxylin (Dako) for 7 min. PBS washes were performed between all steps. The slides were neutralized in ammonia water, dehydrated in graded alcohols (100%, 95%, and 80% ethanol [vol/vol] in H₂O), cleared in xylene and coverslips attached with Permount (Fisher Scientific).

For MART-1 staining, slides were deparaffinized (with xylene), hydrated with distilled water and then placed in citrate buffer (Dako) in a 72° C. oven overnight for antigen retrieval. Following treatment with H₂O₂, slides were incubated in MART-1 primary antibody (1:40, Signet) for 25 min then in LSAB2 biotinylated link antibody (Dako) for 20 min followed by a streptavidin-peroxidase reaction using DAB as a chromogen. Slides were finally counterstained in hematoxylin and then neutralized, dehydrated and coverslips attached as above. Double staining was accomplished by first staining for CD8 (as above) using DAB as the chromogen followed by washing and staining for MART-1 using the alkaline phosphatase system (Leica) omitting the deparaffinization and retrieval steps. Brown staining from the DAB indicated CD8⁺T cells and red staining from the alkaline phosphatase indicated MART1 cells. Both positive and negative controls were stained with the specimens.

Cytotoxicity Assay

CRL-11174 human melanoma cells (ATCC) were cultured in 1 ml of Dulbecco's Modified Eagle Medium (DMEM) (Hyclone, Logan, Utah) supplemented with 10% fetal bovine serum (FBS) (Hyclone, Logan, Utah) and 50 μg/mL gentamicin sulfate (Invitrogen, Carlsbad, Calif.) (2.5×10⁵ cells/well, 6-well plate). DAB/IL2 (Ligand Pharmaceuticals) or PBS was added to the culture (0.05-5 μg/ml) and, after 48 hours, live and dead cells were enumerated by the addition of trypan blue and direct visualization using light microscopy.

### Example 5

### DAB/IL2 Transiently Depletes CD4+, CD8+ and CD4+/CD25+/Foxp3+ T Cells

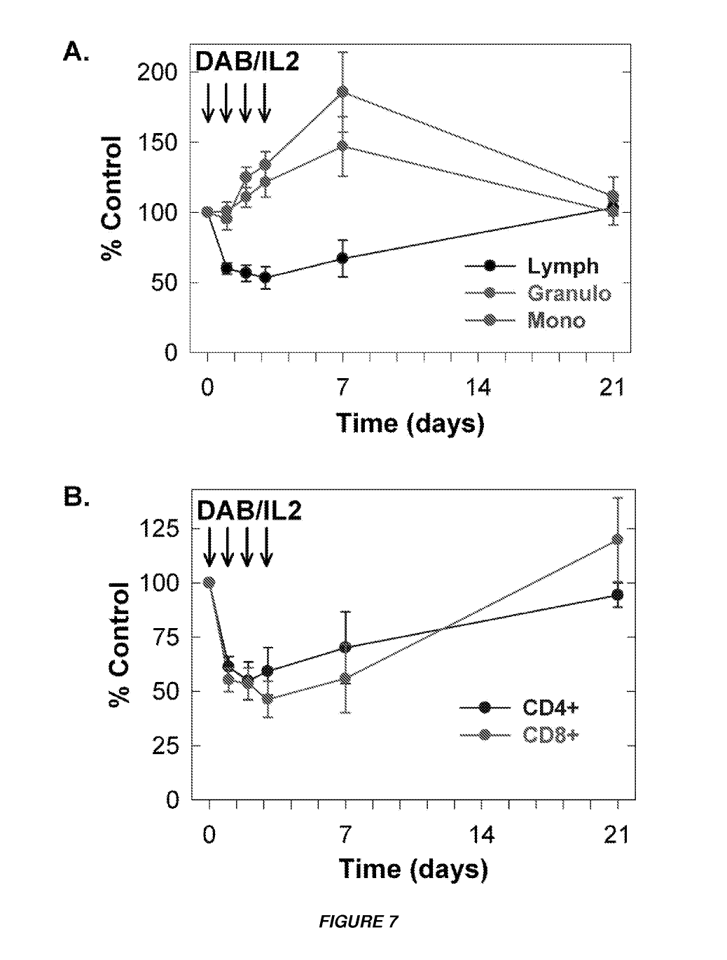

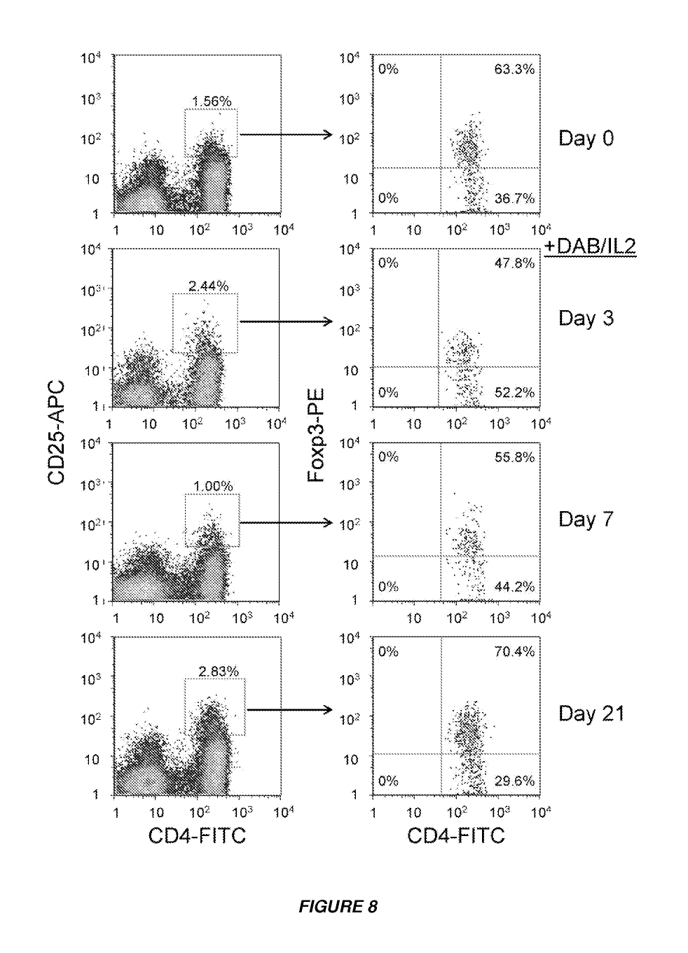

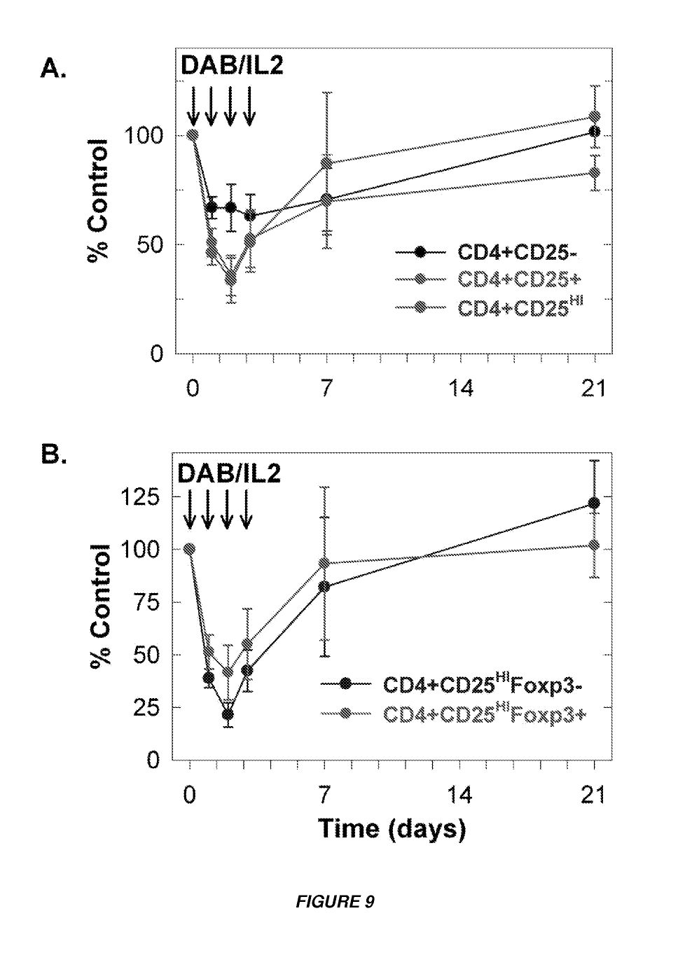

We administered DAB/IL2 (12 μg/kg daily×four days) to stage IV melanoma patients and measured the peripheral blood concentration of lymphocytes, granulocytes and monocytes on days 0, 1, 2, 3, 4, 7 and 21 using an automated hematology analyzer (FIG. 7A; n=10). We observed a reduction in the absolute lymphocyte concentration and an increase in granulocytes and monocytes within 48 hours of DAB/IL2 administration. Next, the absolute concentration of several T cell subsets was quantified by multiplying the percentage of fluorescence-positive cells within the lymphocyte forward/side scatter gate determined using flow cytometric analyses by the absolute lymphocyte concentration determined using an automated hematology analyzer. We quantified the absolute CD4+ and CD8+ T cell concentration in the peripheral blood and found that both T cell subsets were reduced to ˜50% of control within 24-48 hours of DAB/IL2 administration (FIG. 7B; n=10). We co-stained for CD4, CD25 and Foxp3 expression and found that DAB/IL2 depleted the CD4+CD25HIFoxp3+ cell percentage in the lymphocyte gate within 72 hours but that these CD4+CD25HIFoxp3+ cells repopulated the peripheral blood within 21 days (see FIG. 8 for a representative example; CD4+CD25HI cells [left panels] were gated and examined for Foxp3 expression [right panels]). Co-staining for CD4, CD25 and Foxp3 allowed us to gate on several distinct T cell populations and calculate peripheral blood concentrations (based on the absolute lymphocyte concentrations) in order to identify the T cell phenotypes that are most sensitive to DAB/IL2 administration. We were surprised that the peripheral blood CD4+CD25− T cell concentration was depleted by DAB/IL2 albeit to a lesser extent than CD4+CD25+ and CD4+CD25HI T cells (FIG. 9A; n=10; p value <0.05 for each comparison). Given that the targeting mechanism of DAB/IL2 is through CD25, we can only speculate that the depletion of CD4+CD25− cells is due to unidentified indirect consequences of CD4+CD25+ T cell depletion. Interestingly, Foxp3 expression did not significantly alter sensitivity to DAB/IL2 since the depletion of CD4+CD25HIFoxp3+ and CD4+CD25HIFoxp3− was not statistically different (FIG. 9B; p value=0.424).

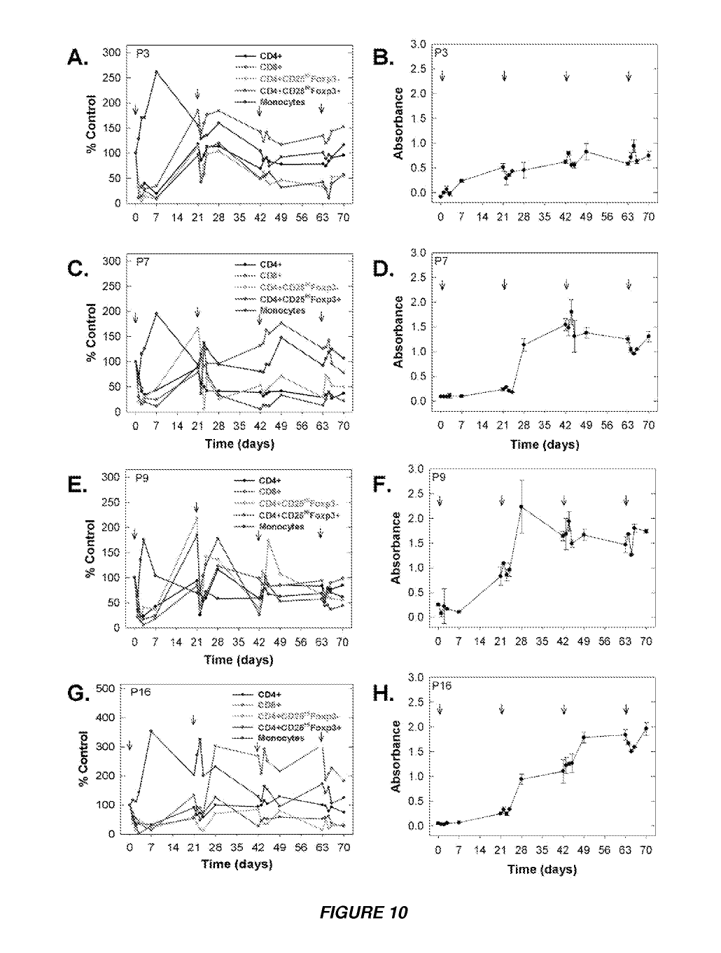

In four patients that received four 3-week cycles (P3, P7, P9 and P16), we found that the T cell depletion and inverse increase in peripheral blood monocytes was blunted in cycles 2-4 of DAB/IL2 (FIGS. 10A, 10C, 10E, and 10G; arrows indicate cycles) and that this effect was coincident with the appearance of anti-DAB/IL2-specific IgG as measured by ELISA (FIGS. 10B, 10D, 10F, and 10H). Additionally, we found that the CD4+CD25HIFoxp3+ T cells were depleted to a greater extent relative to CD8+ T cells after 4 cycles of DAB/IL2 administration (day 70; CD4+CD25HIFoxp3+ T cells=37±16% of control; CD8+ T cells, 128±48% of control; n=4; p value=0.031; FIGS. 10A, 10C, 10E, and 10G). Although the total CD4+ T cell recovery after 4 cycles of DAB/IL2 appeared not to be as robust as that observed by the CD8+ T cells, we observed no statistically significant difference in these T cell subsets (CD4+ T cells, 73±25% of control; CD8+ T cells, 128±48% of control; n=4; p value=0.086).

### Example 6

### T Cell Repopulation After DAB/IL2 Monotherapy is Associated with the De Novo Appearance of Melanoma Antigen-Specific CD8+ T Cells

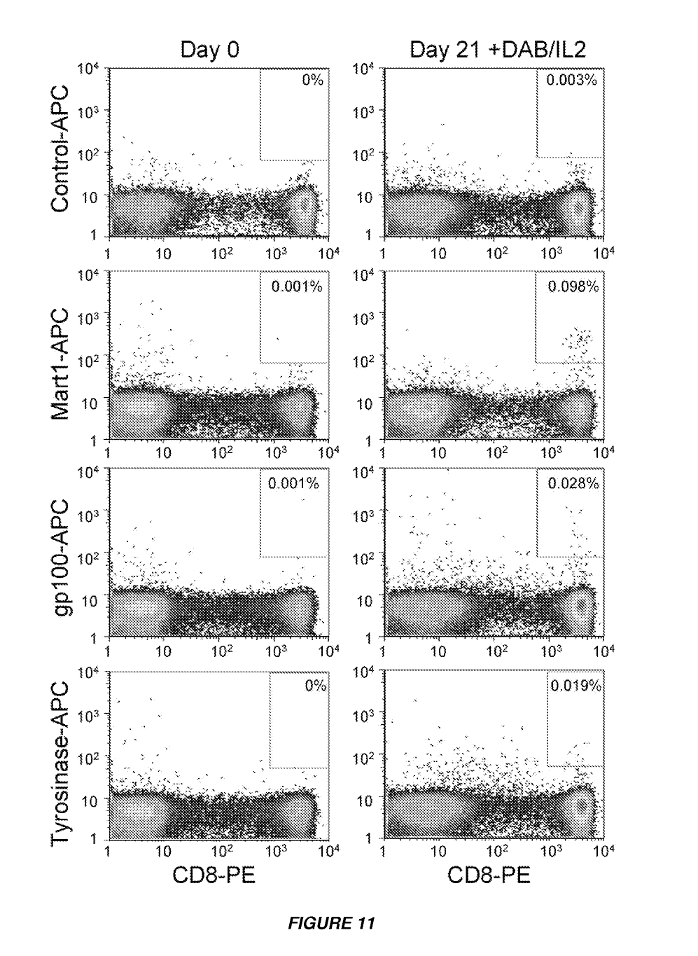

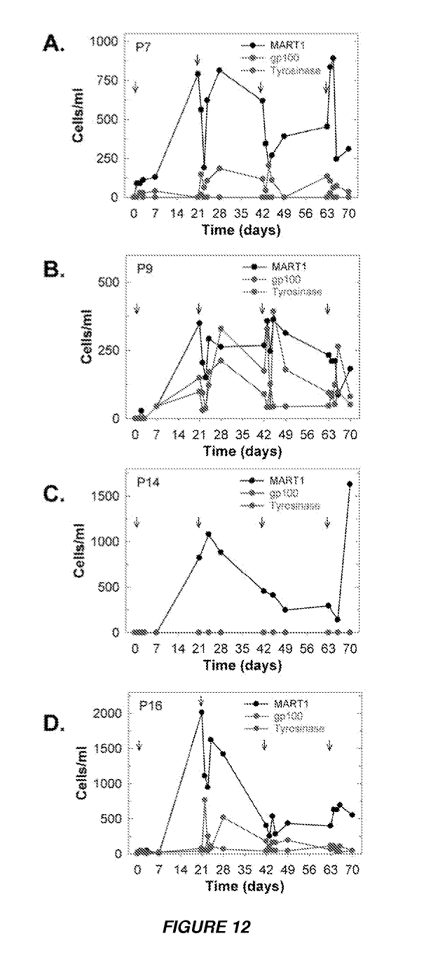

Seven patients expressed the HLA-A2*0201 class I MHC necessary for tetramer-based measurement of CD8+ T lymphocytes specific for the melanoma antigens, MART1, tyrosinase and gp100. We did not detect CD8+ T cells specific for these three melanoma peptide/MHC conjugates prior to DAB/IL2 administration in any of the examined seven patients. However, we did observe the de novo appearance of MART1-specific CD8+ T cells in four HLA-A2*0201+ patients after one cycle of DAB/IL2 as well as CD8+ T cells specific for gp100 and tyrosinase in 2 and 3 patients, respectively (FIGS. 11 and 12). Interestingly, DAB/IL2 transiently decreased the newly detectable MART1-specific CD8+ T cells in three patients at the initiation of cycles 2 and 3 (see FIGS. 12B-12D). These data support the hypothesis that transient depletion of T cells in melanoma patients may disrupt the homeostatic control of cognate immunity and allow for the expansion of effector T cells with specificity against melanoma cells.

### Example 7

### DAB/IL2 Decreases Tumor Burden in Stage IV Melanoma Patients

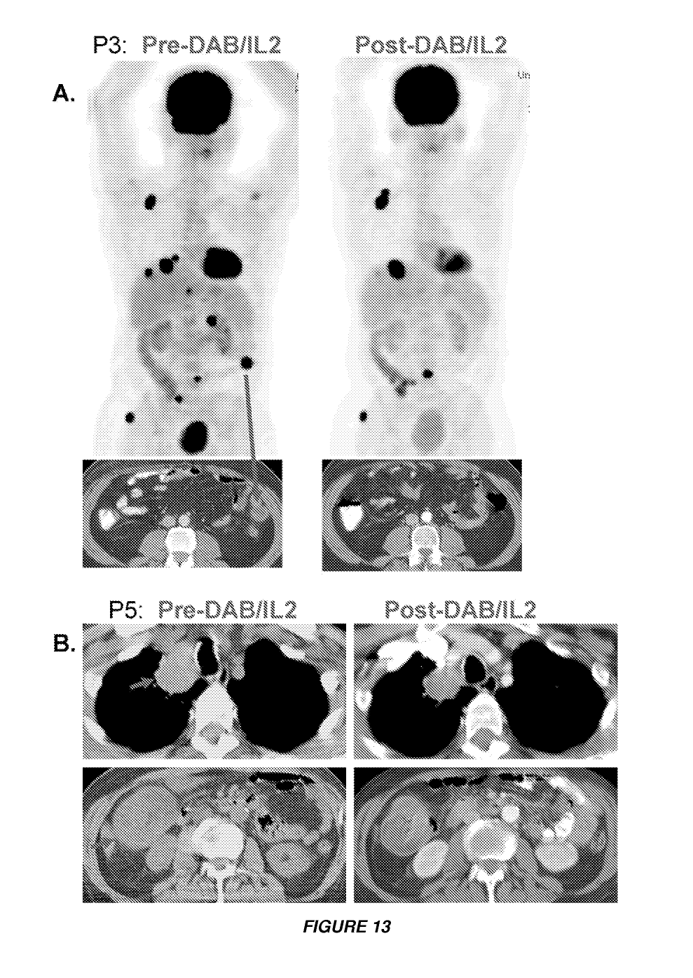

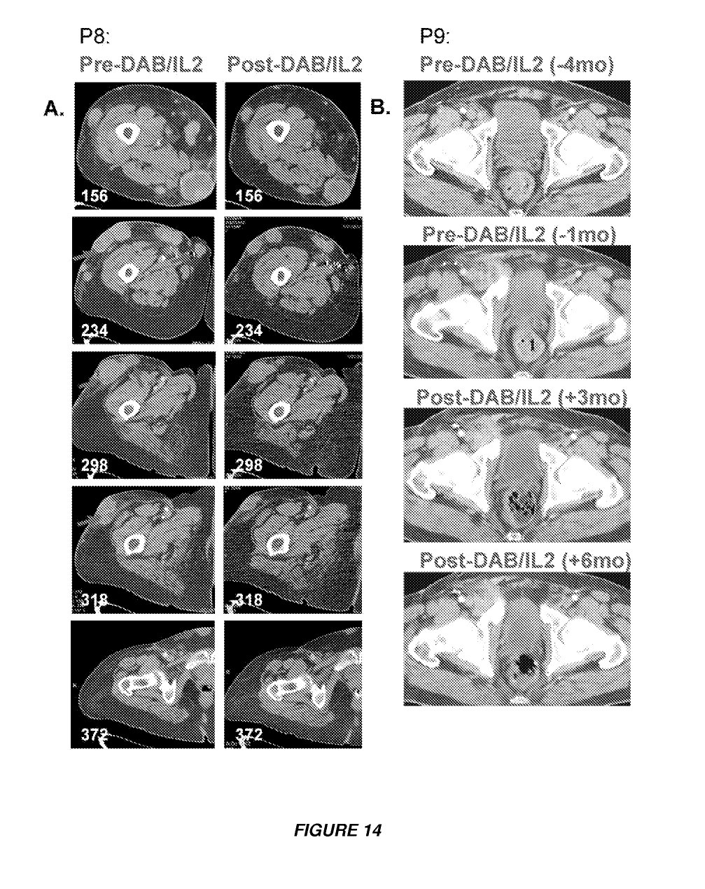

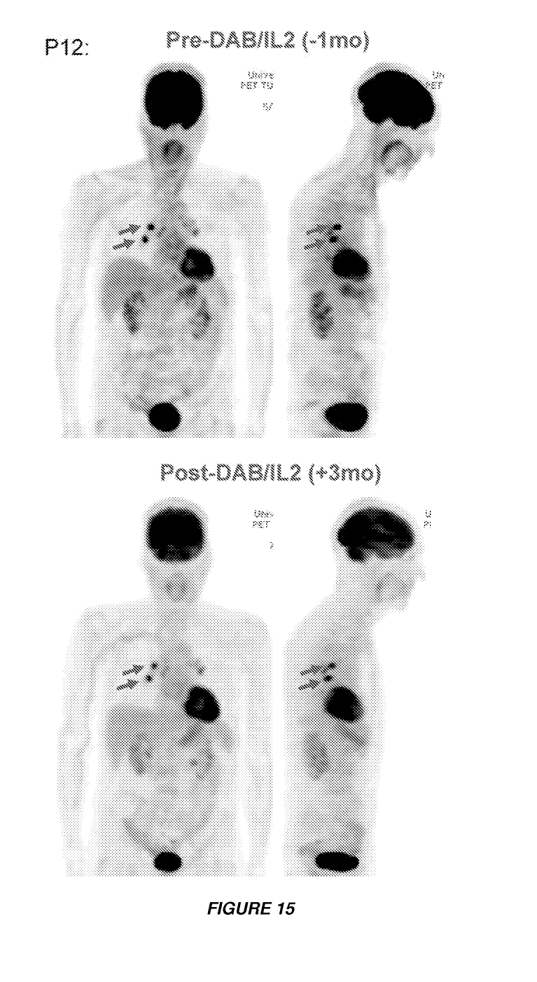

Sixteen heavily pre-treated stage IV melanoma patients were administered 1-4 cycles of DAB/IL2 (12 μg/kg daily×four days every 3 weeks). Positron emission tomography and/or computed tomography were used to evaluate the patients' baseline tumor burden and potential responses three months from initiation of therapy. Table 1 details the characteristics of the patients and the observed responses as per RECIST criteria. We observed reductions in tumor burden in five patients and stabilization of disease in one patient. Patient P3 had developed rapidly progressing subcutaneous, hepatic and mesenteric metastases but after 4 cycles of DAB/IL2, experienced regression of 7 tumors as measured by PET/CT imaging (FIG. 13A). Interestingly, the two largest metastases at baseline grew during treatment. Patient P5 experienced regression of two large melanoma metastases, a right hilar and a right colonic mass (FIG. 13B). Patient P8 had several palpable subcutaneous and intramuscular metastases in her right lower extremity decrease in volume by physical exam after the first cycle of DAB/IL2. She completed four cycles of DAB/IL2, and CT imaging confirmed a decrease in the size of all metastases (FIG. 14A). Patient P9 developed swelling in his right inguinal basin and CT imaging confirmed the development of a large right inguinal mass over a 3 month interval. He experienced decreased swelling after two cycles which was confirmed by CT imaging after a total of four cycles of DAB/IL2 (FIG. 14B). This mass did not change in size for the next 3 months and, after its surgical resection, the patient had no further evidence of disease. The oldest patient enrolled (79 years old; patient P12), developed two right hilar metastases which became less prominent by PET imaging after two cycles of DAB/IL2 (designated Stable Disease; FIG. 15).

TABLE 1
Clinical Outcomes of DAB/IL2 Administration to Melanoma Patients

| | | | | | Adverse | | HLA-A2* | |
|---|---|---|---|---|---|---|---|---|
| ID | Age | M/F | Stage | Cycles | Events (grade) | Outcome(s) | 0201 | CD8+ T Cells |
| P1 | 50 | F | IV | 2 | Erythema (1) | Progressive | − | Not applicable |
| | | | | | | disease | | |
| P2 | 78 | M | IV | 3 | Weakness (1) | Progressive | − | Not applicable |
| | | | | | | Disease | | |
| P3 | 58 | M | IV | 4 | None | Mesenteric & | − | Not applicable |
| | | | | | | Hepatic | | |
| | | | | | | Regressions | | |
| | | | | | | (PR) | | |
| P4 | 66 | F | IV | 1 | None | Progressive | − | Not applicable |
| | | | | | | Disease | | |
| P5 | 72 | M | IV | 2 | Pain at | R. Hilar and | − | Not applicable |
| | | | | | tumor site (1) | Colon | | |
| | | | | | | Regressions | | |
| | | | | | | (PR) | | |
| P6 | 63 | M | IV | 2 | None | Progressive | − | Not applicable |
| | | | | | | Disease | | |
| P7 | 67 | M | IV | 4 | None | Progressive | + | MART1⁺Tyr⁺ |
| | | | | | | Disease | | |
| P8 | 58 | F | IV | 4 | None | IM & SC | − | Not applicable |
| | | | | | | Regressions | | |
| | | | | | | (PR) | | |
| P9 | 46 | M | IV | 4 | None | Inguinal | + | MART1⁺g100⁺ |
| | | | | | | Regression | | Tyr⁺ |
| P10 | 35 | F | IV | 3 | None | Progressive | + | Negative |
| | | | | | | Disease | | |
| P11 | 54 | M | IV | 2 | None | Progressive | + | Negative |
| | | | | | | Disease | | |
| P12 | 79 | M | IV | 2 | Dermatitis (2) | Stable | − | Not applicable |
| | | | | | | Disease | | |
| P13 | 72 | M | IV | 1 | Dehydration (2) | Progressive | − | Not applicable |
| | | | | | | Disease | | |
| P14 | 61 | M | IV | 4 | Vitiligo (1) | Pulmonary, | + | MART1⁺ |
| | | | | | | Hepatic, LN | | |
| | | | | | | & SC | | |
| | | | | | | Regressions | | |
| | | | | | | (PR) | | |
| P15 | 46 | F | IV | 4 | None | Progressive | + | Negative |
| | | | | | | Disease | | |
| P16 | 68 | F | IV | 4 | Arthritis (2) | Progressive | + | MART1⁺g100⁺ |
| | | | | | Dermatitis (1) | Disease | | Tyr⁺ |

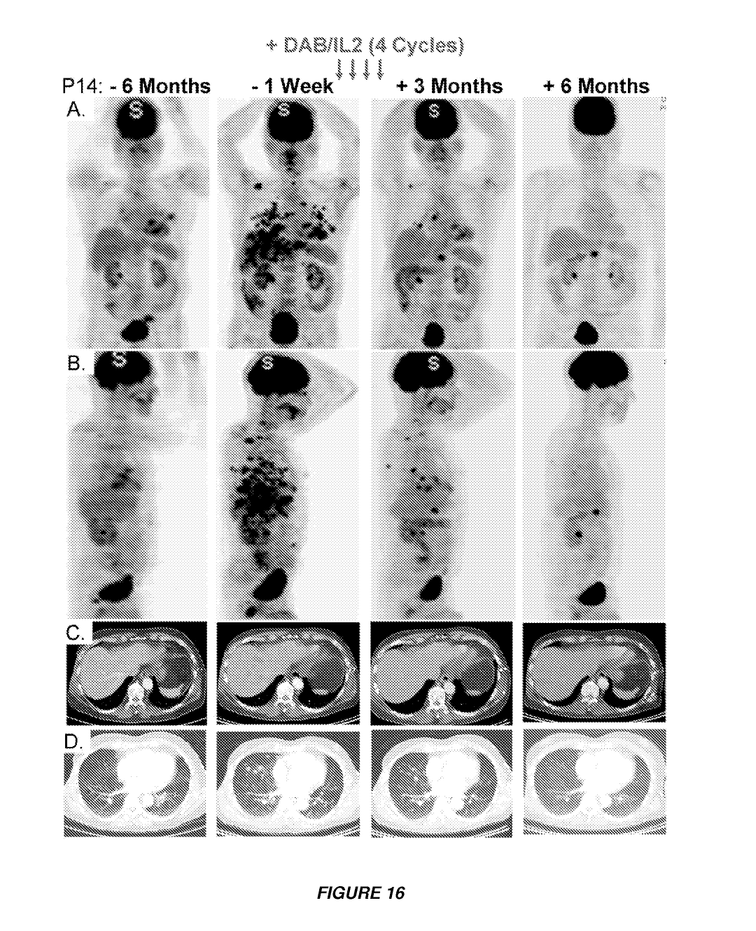

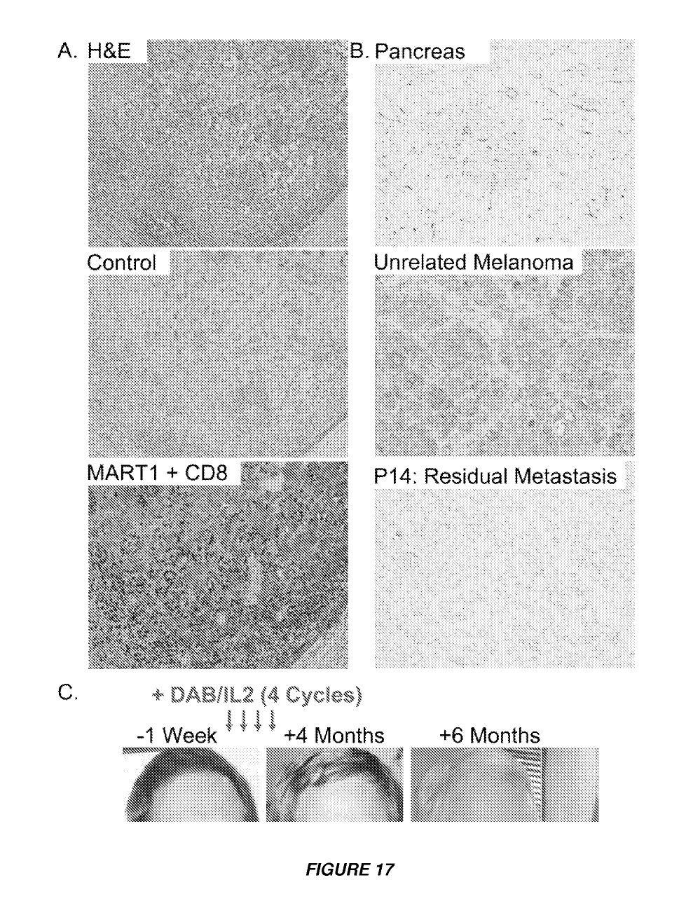

Patient P14 developed widespread melanoma involving the lungs, liver, subcutaneous compartment and adrenal glands over a six month period (FIG. 16). Clinically, he was suffering from appetite and weight loss, fatigue, nausea and shortness of breath. After four cycles of DAB/IL2, PET/CT imaging revealed the complete regression of all hepatic metastases and the majority of pulmonary metastases (FIG. 16). Follow-up PET/CT imaging three months after completion of the fourth cycle of DAB/IL2 demonstrated resolution of the residual pulmonary metastases but a persistently enlarged peri-aortic lymph node. Surgical resection of this residual metastasis was conducted and H&E staining revealed a mononuclear infiltrate within a metastatic melanoma. Double immunohistochemistry for the melanoma protein MART1 and CD8 demonstrated that the melanoma cells (red staining) were surrounded by infiltrating CD8+ T cells (brown staining; FIG. 17A) but not CD4+ T cells. These remaining melanoma cells were completely devoid of HLA-A, B or C expression as determined using a monoclonal antibody specific for a non-polymorphic portion of these HLA molecules (FIG. 17B; compare positive controls, pancreas and unrelated melanoma, to P14 residual metastasis). Coincident with this objective response, the patient reported decreased pigmentation in his hair and skin consistent with the development of vitiligo, an autoimmune disease against melanocytes (FIG. 17C).

We were surprised by the high partial response rate in these 16 patients and postulated that DAB/IL2 may exhibit direct cytotoxic effects against human melanoma cells. However, exposure of DAB/IL2 to proliferating human melanoma cells in vitro at a concentration 15-fold higher than the obtainable peak plasma concentration of DAB/IL2 in humans (0.3 μg/ml) had no effect on cell viability or proliferation (0.05-5 μg/ml×48 hours; vehicle control, 7.12±0.13×10⁵ cells; +5 μg/ml DAB/IL2, 7.35±0.37×10⁵ cells; p value=0.444).

### Discussion of Examples 5-7

Melanoma incidence has risen by 25-31% over the last decade and is now the 5th most common cancer in men and the 6th most common cancer in women [McDermott et al. (2000)]. Melanoma causes a disproportionate mortality in young and middle-aged individuals and, as such, displays one of the highest “loss of potential life” rates among the adult-onset cancers (18.6 years per melanoma-related death). In the United States, over 8000 adults die of melanoma annually, and 84% of melanoma patients with distant metastases succumb to their disease within 5 years of diagnosis.

The treatment options for patients with metastatic melanoma are limited to palliation or to aggressive therapy with high dose IL-2 or biochemotherapy using cisplatin, vinblastine, dacarbazine, IL-2 and interferon α-2b. The response rate to high dose IL-2 is low (16%) but durable cures have been observed in approximately 6-10% of the patients that can tolerate the systemic toxicity (i.e. hypotension, capillary leak syndrome, sepsis and renal failure). Although biochemotherapy has been reported to yield a 35-50% partial response rate and up to a 20% complete response rate, median survival duration is only 12.2 months [McDermott et al. (2000); Legha et al. (1998)]. Early published reports of clinical trials of humanized anti-CTLA4 monoclonal antibodies have indicated a 10-20% partial response rate in melanoma patients [Peggs et al. (2006)]. In the current study, we observed a 31% partial response rate after treatment with DAB/IL2 (5/16 patients) which is clinically significant given the low toxicity of this agent. Importantly, the majority of patients who are treated with high dose IL-2, biochemotherapy and/or anti-CTLA4 ultimately experience progression and few efficacious alternative treatments are currently available.

We found that transient depletion of CD4+ and CD8+ T cells in melanoma patients via targeting of IL-2 receptor-expressing cells resulted in T cell repopulation in the peripheral blood and the de novo appearance of CD8+ T cells with specificity for melanoma antigens (in 4/7 HLA-A2*0201 patients). We had anticipated that the detection of peripheral blood MART1-, gp100- and tyrosinase-specific CD8+ T cells in these HLA-A2*0201+ patients might correlate with tumor regressions. The three HLA-A2*0201+ patients who did not develop any detectable MART1-, gp100- and tyrosinase-specific CD8+ T cells also did not experience regression of their melanoma metastases (Table 1). However, we observed the regression of melanoma metastases in only 2/4 HLA-A2*0201+ patients who developed melanoma antigen-specific CD8+ T cells (Table 1). We can only speculate that the two patients who developed melanoma antigen-specific CD8+ T cells but did not experience tumor regressions may have melanomas that express low class I MHC or effector CD8+ T cells that are compromised by low affinity T cell receptors and/or the tumor microenvironment. Importantly, the peptide/MHC tetramers used in this study can only detect a miniscule fraction of the possible CD8+ T cells that have specificity for MART1, gp100, tyrosinase or other melanoma antigens.

Intriguingly, patients P3 and P14 experienced the regression of multiple metastastic melanomas simultaneously with the persistence and even growth of other metastatic melanomas (i.e. a mixed response). The residual peri-aortic mass in patient P14 was confirmed to express the melanoma antigen, MART1, and this patient developed peripheral blood MART1-specific CD8+ T cells within 21 days of transient T cell depletion. Despite immunohistochemical evidence that CD8+ T cells appeared to surround the MART1+ melanoma cells, this residual metastatic melanoma was not cleared. Interestingly, the melanoma cells did not express the class I MHC proteins HLA-A, B or C which may partly explain the lack of regression of this particular metastasis. We suspect that differences in melanoma antigen expression and/or additional tumor immunoevasion tactics also may explain such differential anti-tumor effects within a single host and future studies will be directed at further examination of the phenotypes of melanoma cells and infiltrating immune cells in growing and regressing melanomas within a single host.

DAB/IL2 administration transiently decreased CD4+CD25−, CD4+CD25+, CD4+CD25HIFoxp3−, CD4+CD25HIFoxp3+, CD8+ T cells and, in certain patients, melanoma antigen-specific CD8+ T cells. These data suggest that DAB/IL2 is not selectively cytotoxic to T regulatory cells which may be due, in part, to the high IL-2 receptor expression of activated effector T cells. We found that all examined T cell subsets repopulated the peripheral blood and presume that this repopulation is due either to a proliferative expansion or re-trafficking of T cells from lymph nodes. Interestingly, CD4+ or CD8+ T cell depletion in mice has been found to cause a proliferative expansion of the residual T cells that restores the original T cell pool size [Wu et al. (2004)]. This peripheral expansion has been termed homeostatic proliferation and can prevent the induction of tolerance to transplanted organs and cause anti-tumor responses against melanomas and colon cancer in mice [Wu et al. (2004); Dummer et al. (2002); Hu et al. (2002)]. Although the mechanisms for these effects are not well established, homeostatic proliferation of CD4+ and CD8+ T cells is, in part, driven by MHC/peptide recognition. We postulate that transient T cell depletion in cancer patients may cause a rebound expansion of T cells with a shifted TCR repertoire that includes increased melanoma antigen-specific CD8+ T cells.

In conclusion, we have demonstrated that T cell depletion with DAB/IL2 in melanoma patients is followed by a T cell repopulation of the peripheral blood, the de novo appearance of CD8+ T cells specific for melanocyte differentiation antigens and regression of melanoma metastases. Last, we anticipate that the limited or pulsed administration of alternative T cell depleting agents may prove useful for the activation of cognate immunity against neoplastic cells in cancer patients.

### REFERENCES

Atkins M B, Lotze M T, Dutcher J P, Fisher R I, Weiss G, Margolin K, Abrams J, Sznol M, Parkinson D, Hawkins M et al: High-dose recombinant interleukin 2 therapy for patients with metastatic melanoma: analysis of 270 patients treated between 1985 and 1993. J Clin Oncol 1999, 17(7):2105-2116.

Attia P, Maker A V, Haworth L R, Rogers-Freezer L, Rosenberg S A: Inability of a fusion protein of IL-2 and diphtheria toxin (Denileukin Diftitox, DAB₃₈₉IL-2, ONTAK) to eliminate regulatory T lymphocytes in patients with melanoma. J Immunother 2005, 28(6):582-592.

Barnett B, Kryczek I, Cheng P, Zou W, Curiel T J: Regulatory T cells in ovarian cancer: biology and therapeutic potential. Am J Reprod Immunol 2005, 54(6):369-377.

Beilharz M W, Sammels L M, Paun A, Shaw K, van Eeden P, Watson M W, Ashdown M L: Timed ablation of regulatory CD4+ T cells can prevent murine AIDS progression. J Immunol 2004, 172(8):4917-4925.

Berger C L, Tigelaar R, Cohen J, Mariwalla K, Trinh J, Wang N, Edelson R L: Cutaneous T-cell lymphoma: malignant proliferation of T-regulatory cells. Blood 2005, 105(4)1 640-1647.

Boon T, Coulie P G, Van den Eynde B J, van der Bruggen P: Human T cell responses against melanoma. Annu Rev Immunol 2006, 24:175-208.

Dannull J, Su Z, Rizzieri D, Yang B K, Coleman D, Yancey D, Zhang A, Dahm P, Chao N, Gilboa E et al: Enhancement of vaccine-mediated antitumor immunity in cancer patients after depletion of regulatory T cells. J Clin Invest 2005, 115(12):3623-3633.

Dummer W, Niethammer A G, Baccala R, Lawson B R, Wagner N, Reisfeld R A, Theofilopoulos A N: T cell homeostatic proliferation elicits effective antitumor autoimmunity. J Clin Invest 2002, 110(2):185-192.

Ehrenstein M R, Evans J G, Singh A, Moore S, Warnes G, Isenberg D A, Mauri C: Compromised function of regulatory T cells in rheumatoid arthritis and reversal by anti-TNFalpha therapy. J Exp Med 2004, 200(3):277-285.

Frankel A E, Surendranathan A, Black J H, White A, Ganjoo K, Cripe L D: Phase II clinical studies of denileukin diftitox diphtheria toxin fusion protein in patients with previously treated chronic lymphocytic leukemia. Cancer 2006, 106(10):2158-2164.

Hu H M, Poehlein C H, Urba W J, Fox B A: Development of antitumor immune responses in reconstituted lymphopenic hosts. Cancer Res 2002, 62(14):3914-3919.

Jemal A, Murray T, Ward E, Samuels A, Tiwari R C, Ghafoor A, Feuer E J, Thun M J: Cancer statistics, 2005. CA Cancer J Clin 2005, 55(1):10-30.

Jones E, Dahm-Vicker M, Golgher D, Gallimore A: CD25+ regulatory T cells and tumor immunity. Immunol Lett 2003, 85(2):141-143.

Jones E, Dahm-Vicker M, Simon A K, Green A, Powrie F, Cerundolo V, Gallimore A: Depletion of CD25+ regulatory cells results in suppression of melanoma growth and induction of autoreactivity in mice. Cancer Immun 2002, 2:1.

Keilholz U, Conradt C, Legha S S, Khayat D, Scheibenbogen C, Thatcher N, Goey S H, Gore M, Dorval T, Hancock B et al: Results of interleukin-2-based treatment in advanced melanoma: a case record-based analysis of 631 patients. J Clin Oncol 1998, 16(9):2921-2929.

Kirkwood J M, Strawderman M H, Ernstoff M S, Smith T J, Borden E C, Blum R H: Interferon alfa-2b adjuvant therapy of high-risk resected cutaneous melanoma: the Eastern Cooperative Oncology Group Trial EST 1684. J Clin Oncol 1996, 14(1):7-17.

Legha S S, Ring S, Eton O, Bedikian A, Buzaid A C, Plager C, Papadopoulos N: Development of a biochemotherapy regimen with concurrent administration of cisplatin, vinblastine, dacarbazine, interferon alfa, and interleukin-2 for patients with metastatic melanoma. J Clin Oncol 1998, 16(5):1752-1759.

McDermott D F, Mier J W, Lawrence D P, van den Brink M R, Clancy M A, Rubin K M, Atkins M B: A phase II pilot trial of concurrent biochemotherapy with cisplatin, vinblastine, dacarbazine, interleukin 2, and interferon alpha-2B in patients with metastatic melanoma. Clin Cancer Res 2000, 6(6):2201-2208.

Onizuka S, Tawara I, Shimizu J, Sakaguchi S, Fujita T, Nakayama E: Tumor rejection by in vivo administration of anti-CD25 (interleukin-2 receptor alpha) monoclonal antibody. Cancer Res 1999, 59(13):3128-3133.

Peggs K S, Quezada S A, Korman A J, Allison J P: Principles and use of anti-CTLA4 antibody in human cancer immunotherapy. Curr Opin Immunol 2006, 18(2):206-213.

Shimizu J, Yamazaki S, Sakaguchi S: Induction of tumor immunity by removing CD25+CD4+ T cells: a common basis between tumor immunity and autoimmunity. J Immunol 1999, 163(10):5211-5218.

Sutmuller R P, van Duivenvoorde L M, van Elsas A, Schumacher T N, Wildenberg M E, Allison J P, Toes R E, Offringa R, Melief C J: Synergism of cytotoxic T lymphocyte-associated antigen 4 blockade and depletion of CD25(+) regulatory T cells in antitumor therapy reveals alternative pathways for suppression of autoreactive cytotoxic T lymphocyte responses. J Exp Med 2001, 194(6):823-832.

Thompson J F, Scolyer R A, Kefford R F: Cutaneous melanoma. Lancet 2005, 365(9460):687-701.

Turk M J, Guevara-Patino J A, Rizzuto G A, Engelhorn M E, Sakaguchi S, Houghton A N: Concomitant tumor immunity to a poorly immunogenic melanoma is prevented by regulatory T cells. J Exp Med 2004, 200(6):771-782.

Viguier M, Lemaitre F, Verola O, Cho M S, Gorochov G, Dubertret L, Bachelez H, Kourilsky P, Ferradini L: Foxp3 expressing CD4+CD25(high) regulatory T cells are overrepresented in human metastatic melanoma lymph nodes and inhibit the function of infiltrating T cells. J Immunol 2004, 173(2):1444-1453.

Wu Z, Bensinger S J, Zhang J, Chen C, Yuan X, Huang X, Markmann J F, Kassaee A, Rosengard B R, Hancock W W, Sayegh M H, Turka L A: Homeostatic proliferation is a barrier to transplantation tolerance. Nat Med 2004, 10(1):87-92.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of treating or reducing the risk of recurrence of a cancer in a subject, comprising administering an effective amount of a T cell depleting composition to the subject.

2. The method of claim 1, wherein the cancer is a cancer other than a T cell lymphoma.

3. The method of claim 1, wherein the cancer is a melanoma or a breast cancer.

4. The method of claim 1, wherein the T cell depleting composition is administered parenterally.

5. The method of claim 4, wherein T cell depleting composition is administered intravenously.

6. The method of claim 1, wherein the T cell depleting composition comprises methotrexate, busulfan, cyclophosphamide, fludarabine, FTY720, an anti-CD3 antibody, an IL-2-cell toxin fusion protein, or combinations thereof.

7. The method of claim 6, wherein the T cell depleting composition comprises the IL-2-cell toxin fusion protein, and wherein the cell toxin is a diphtheria toxin.

8. The method of claim 7, wherein the T cell depleting composition comprises DAB₃₈₉IL-2.

9. The method of claim 8, wherein the effective amount of DAB₃₈₉IL-2 administered is about 12 mcg/kg.

10. The method of claim 9, wherein the DAB₃₈₉IL-2 is administered in a cycle of once daily for four days, and wherein the cycle is repeated about every 21 days for at least three cycles.

11. The method of claim 1, wherein the subject is human.

12. A method of stimulating an immune response against a cancer in a subject, comprising:
(a) depleting T cells in the subject; and
(b) permitting rebounding of T cells in the subject to thereby stimulate an immune response against the cancer.

13. The method of claim 12, comprising repeating the depleting and the permitting of rebounding of T cells in the subject a desired number of times.

14. The method of claim 12, wherein depleting T cells comprises depleting total T cell counts in the subject.

15. The method of claim 12, wherein the cancer is a cancer other than a T cell lymphoma.

16. The method of claim 12, wherein the cancer is a melanoma or a breast cancer.

17. The method of claim 12, wherein depleting T cells in the subject comprises administering an effective amount of a T cell depleting composition to the subject.

18. The method of claim 17, wherein the T cell depleting composition is administered parenterally.

19. The method of claim 18, wherein T cell depleting composition is administered intravenously.

20. The method of claim 17, wherein the T cell depleting composition comprises methotrexate, busulfan, cyclophosphamide, fludarabine, FTY720, an anti-CD3 antibody, an IL-2-cell toxin fusion protein, or combinations thereof.

21. The method of claim 20, wherein the T cell depleting composition comprises the IL-2-cell toxin fusion protein, and wherein the cell toxin is a diphtheria toxin.

22. The method of claim 21, wherein the T cell depleting composition comprises DAB₃₈₉IL-2.

23. The method of claim 22, wherein the effective amount of DAB₃₈₉IL-2 administered is about 12 mcg/kg.

24. The method of claim 23, wherein the DAB₃₈₉IL-2 is administered in a cycle of once daily for four days, and wherein the cycle is repeated about every 21 days for at least three cycles.

25. The method of claim 12, wherein the subject is human.
